(19) United States
(12) Patent Application Publication
(10) Pub. No.: US 2011/0125230 A1
(43) Pub. Date: May 26, 2011

(54) Portable Light Therapy Devices

(76) Inventors: Ronald Charles Friedman, Cleveland, OH (US); Kingman P. Strohl, Shaker Heights, OH (US); Phyllis C. Zee, Oak Park, IL (US)

(21) Appl. No.: 12/623,568

(22) Filed: Nov. 23, 2009

(51) Int. Cl. A61N 5/06

(52) U.S. Cl. 607/90

(57) ABSTRACT

A light therapy device includes a first module adapted to be coupled to a head of an individual, and a second module including at least one light, the second module being coupled to the first module so that the second module is positioned relative to the individual's eyes, and the second module powering the light to provide indirect light therapy to the individual.

### BACKGROUND

Circadian rhythms are cyclical biological changes that occur regularly over a 24-hour cycle. Such rhythms can dictate human processes and behaviors, such as body core temperature, heart rate, digestion, and sleep cycles, to name a few. Circadian rhythms can be altered by environmental conditions, such as sleeplessness, travel, and seasonal affective disorder.

Techniques to recover from such ailments can include the use of light therapy. The application of light to an individual's eyes is believed to be one of the strongest cues for entrainment. However, existing devices that are used for light therapy can be bulky, inconvenient, and time consuming. For example, a typical light therapy device includes a bank of lights in a housing that is self-contained or placed on a table. The individual is forced to remain stationary and focused on the lights for a period of time (e.g., 15-45 minutes) during the therapy. Such a device can be both inconvenient and uncomfortable.

### SUMMARY

In one aspect, a light therapy device includes a first module adapted to be coupled to a head of an individual, and a second module including at least one light, the second module being coupled to the first module so that the second module is positioned relative to the individual's eyes, and the second module powering the light to provide indirect light therapy to the individual.

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 is a perspective view of an example light therapy device.

FIG. 2 is an exploded view of the light therapy device of FIG. 1.

FIG. 3 is a front view of the light therapy device of FIG. 1.

FIG. 4 is a side view of the light therapy device of FIG. 1.

FIG. 5 is a top view of the light therapy device of FIG. 1.

FIG. 6 is a front view of an example light therapy module of the light therapy device of FIG. 1.

FIG. 7 is a perspective cross-sectional view taken along line 7-7 of the light therapy module of FIG. 6.

FIG. 8 is a schematic cross-section view taken along line 7-7 of the light therapy module of FIG. 6.

FIG. 9 is a perspective view of the light therapy device of FIG. 1 worn by an individual.

FIG. 10 is a side view of the light therapy device and individual of FIG. 9.

FIG. 11 is a front view of the light therapy device and individual of FIG. 9.

### DETAILED DESCRIPTION

Examples described herein are directed to light therapy devices. In some examples, the devices provide an efficient, low-brightness, indirect reflector which conceals the light source from the eye and delivers a therapeutic dose while minimizing glare, visual hot-spots, or discomfort to the wearer. The devices use lights having a spectrum or spectral range that can influence circadian rhythm, alertness, arousal, and sleep cycle and mitigate a number of medical conditions including insomnia due to a disorder in circadian rhythm, seasonal affective disorder, jet lag, and any other disorders in which light exposure is known to have a therapeutic effect.

The example devices provide light from a concealed source, minimizing glare, visual discomfort, or direct danger to the eye from a high light intensity or heat. The devices can be electrically or battery powered (including rechargeable), are lightweight and portable, and provide light as a treatment without confinement or immobilizing the individual by restricting the individual's other activities, such as exercise, computer use, office work, reading, or other visual tasks.

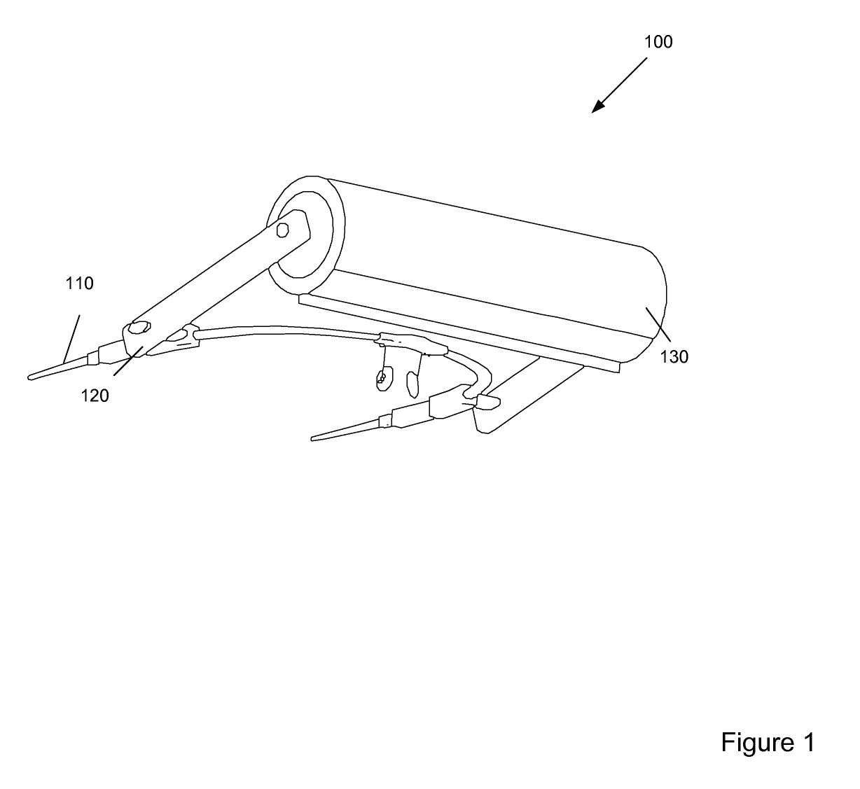

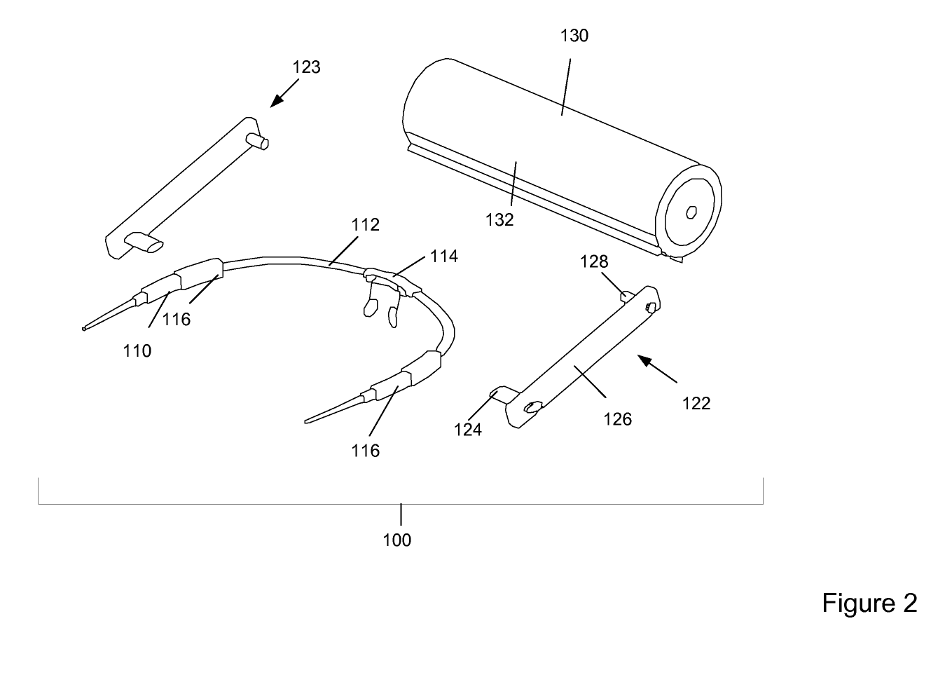

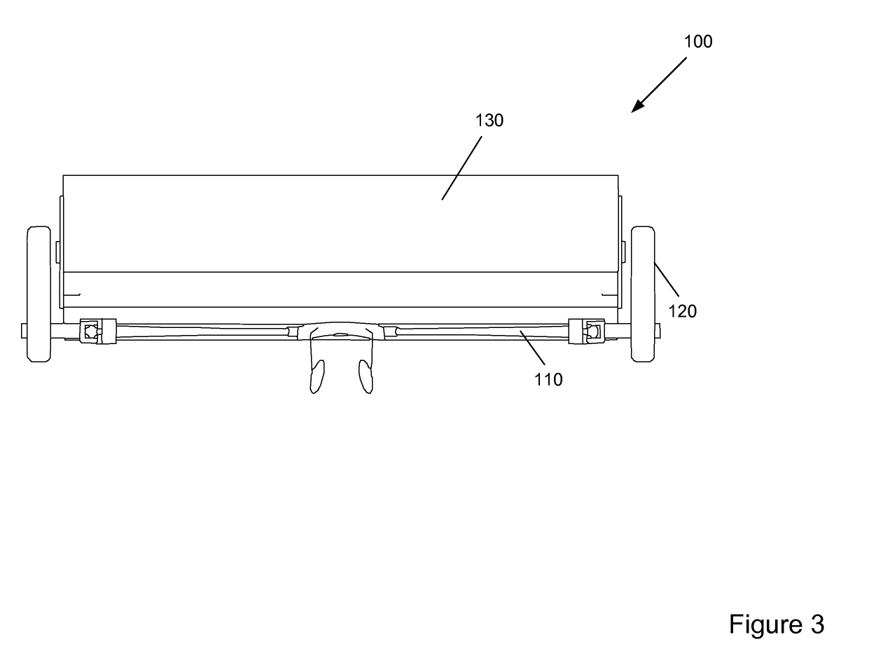

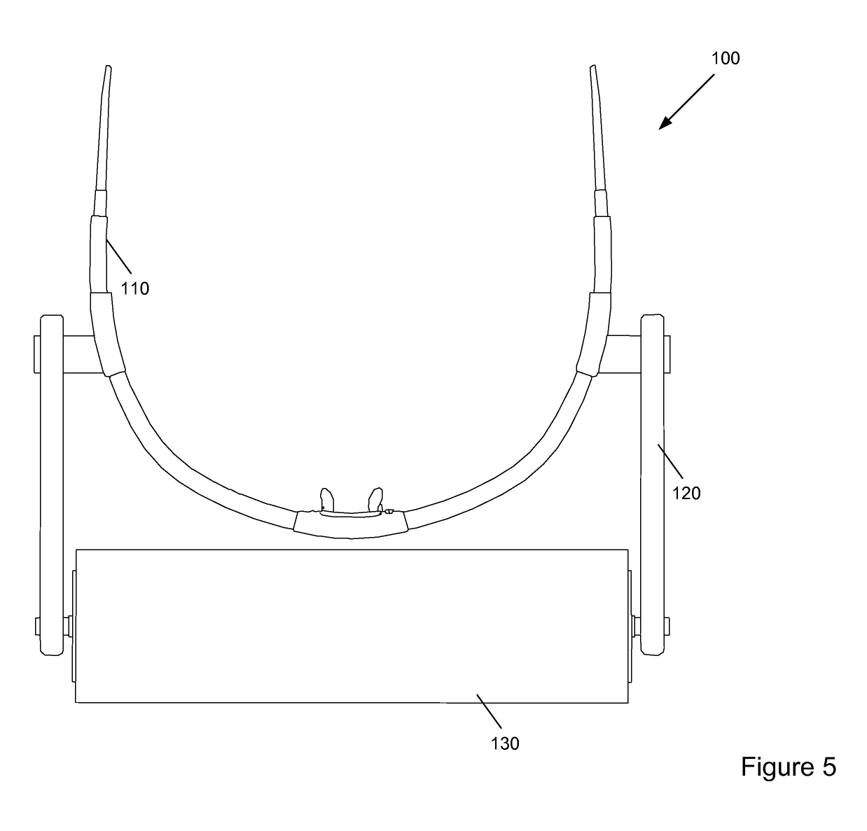

Referring now to FIGS. 1-5, an example device 100 is shown. The device includes a frame 110, a member 120, and a therapy module 130.

The frame 110 is a support structure for the therapy module 130 that allows the therapy module 130 to be coupled to the individual's head. In one non-limiting example, the frame 110 is sized to be worn on the individual's face. The frame 110 includes a frame front 112, a bridge 114, and end pieces 116. The bridge 114 is sized to be placed on the individual's nose, and the end pieces 116 extend to rest on the individual's ears. The frame front 112 typically does not include any lenses, since the frame 110 is used to hold the therapy module 130 in place, rather than being used for visual correction. Although, in alternative embodiments, corrective and/or tinted/polarized lenses can be included in the front frame 112.

The member 120 extends from the frame 110 and allows the therapy module 130 to be pivoted relative to the frame 110. Specifically, the member 120 includes a member 122, which includes a member 124 coupled to the frame 110, a member 126 coupled to the member 124, and a member coupled between the member 126 and the therapy module 130. A member 123 positioned on the opposite side of the frame 110 is configured in a similar manner.

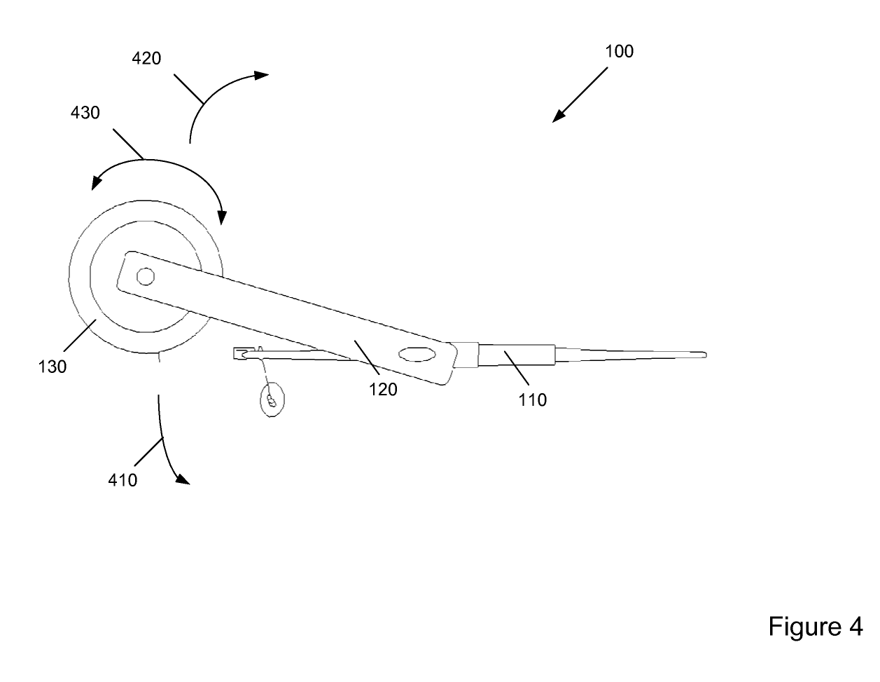

In example embodiments, one or both of the members 124, 128 pivot relative to the member 126 so that the therapy module 130 can be moved generally up and down in directions 410, 420 relative to the frame 110. See FIG. 4. In addition, the therapy module 130 can be rotated about the member 128. In this manner, the light therapy provided by the therapy module 130 can be adjusted, as described further below.

The therapy module 130 includes a cylindrical body 132 in which one or more light modules are positioned, as described below. In use, the cylindrical body 132 emits light that is directed to the individual's eyes when the device 100 is worn.

Beneficial light therapy can be provided through adjustment of the type of light, intensity of the light, and position of the light therapy module 130 relative to the individual's eyes. Since the device 100 is worn on the individual's face, this therapy is provided without immobilizing the individual. The individual can wear the device and receive the therapy while at the same time performing typical functions like exercise, computer use, office work, reading, or other visual tasks.

In example embodiments, the cylindrical body 132 of the therapy module 130 is made of a reflective material, such as specular alzak aluminum. Other types of materials can also be used.

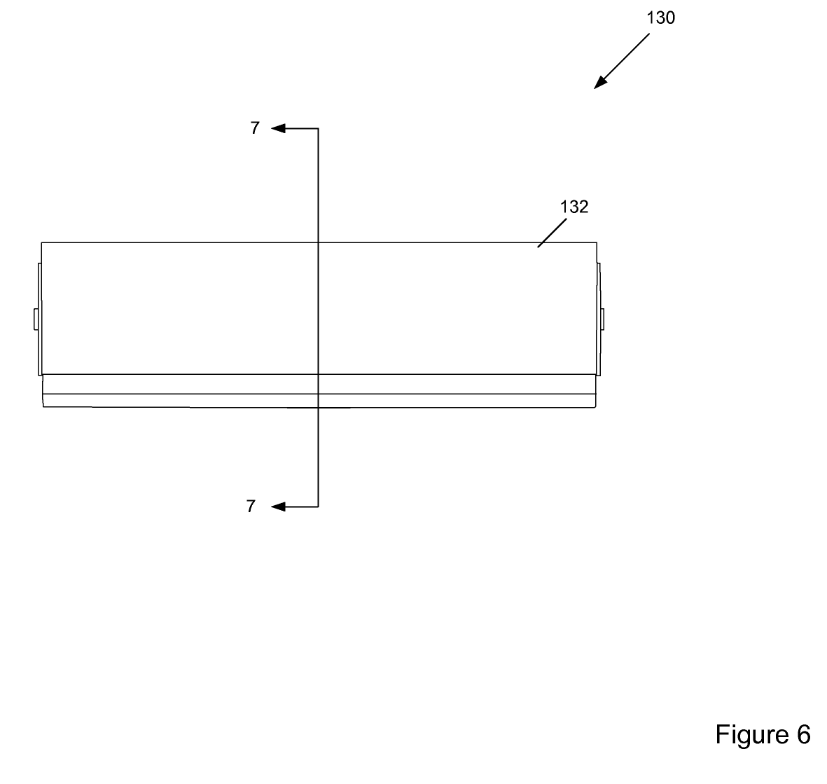

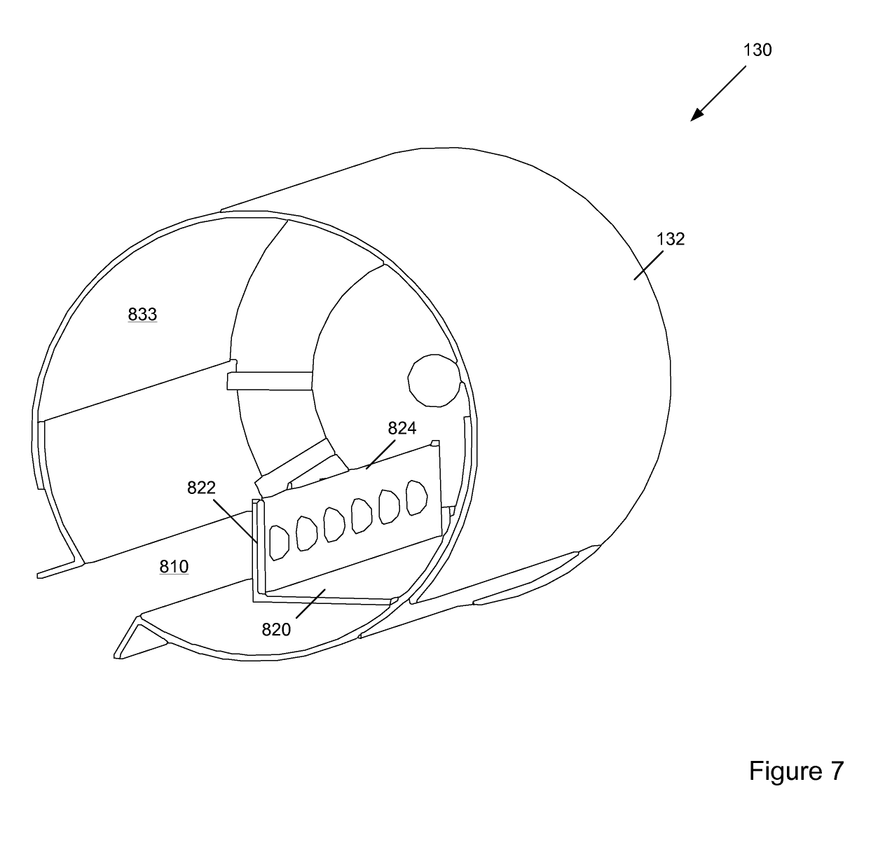

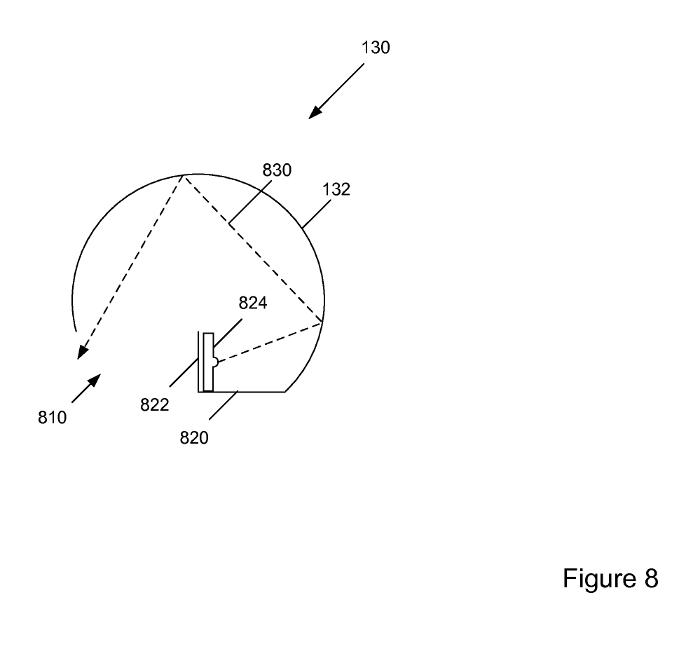

Referring now to FIGS. 6-8, additional views of the therapy module 130 are shown. The cylindrical body 132 of the therapy module 130 includes members 820, 822, and an opening 810. In the example shown, the cylindrical body 132 is generally C-shaped to form the opening 810.

A light-emitting diode (LED) bank 824 is coupled to the member 820. In example embodiments, the LED bank 824 can include one or more LEDs. The LEDs can be a single color (e.g., blue or white) or multi-colored (e.g., one-half blue and one-half white).

In example embodiments, the LEDs are selected to include blue (460-477 nm) LEDs. In other examples, the LEDs can be green and full spectra, or any combination of LED-colored light sources. Other light colors and types of light sources can also be used.

In the example shown, light emitted by the LED bank 824 is projected against an interior 833 of the cylindrical body 132 of the therapy module 130. The light is reflected one or more times by the cylindrical body 132 until the light exits the opening 810 and is directed into the eyes of the individual. One example of such an indirect light path 830 is shown.

Other configurations for the therapy module 130 are possible. For example, multiple LEDs can be mounted at different points on the cylindrical member 132 to adjust an amount of direct versus indirect light that is provided through the opening 810. In other examples, the shape of the cylindrical member 132 can be modified. For example, the curve of the member 132 can be modified to increase or decrease a number of reflections for the light as the light is reflected out of the opening 810.

In example embodiments, the LED bank 824 is powered by one or more batteries. For example, one or more batteries can be located within the device 100, or can be provided on a separate battery pack. For example, the batteries can be contained within a battery back that is worn on the individual's belt. A cord can be extended from the battery pack to the therapy module to power the LED bank 824. Other configurations are possible.

In example embodiments, the cylindrical member 132 of the therapy module 130 is sized to extend generally across the brow of the individual. For example, the cylindrical member 132 can be approximately 5-7 inches in length, less then or equal to 7 inches in length, less than or equal to 6 inches in length, or less than or equal to 5 inches in length. The cylindrical member 132 can have a diameter of approximately 1.5-1.75 inches, less than or equal to 1.75 inches, or less than or equal to 1.5 inches.

The device can be used as follows. Initially, the frame 110 is positioned on the individual's face so that the bridge 114 is on the individual's nose and the end pieces 116 are held by the individual's ears. Next, the position of the therapy module 130 relative to the individual's eyes is adjusted. This can include moving the relative height of the therapy module 130, as well as rotating the therapy module 130 to adjust the position of the opening 810.

Once the therapy module 130 is positioned as desired, the LED bank 824 is energized so that light is directed through the opening 810 and into the eyes of the individual. This light provides the therapy. Since the therapy module 130 can be positioned above the line-of-sight of the individual, and the therapy light is typically indirect light, the individual is able to see while therapy is provided. This allows the individual to continue normal tasks, such as exercise, computer use, office work, reading, or other visual tasks. Once therapy is complete, the LED bank 824 is turned off, and the device 100 can be removed.

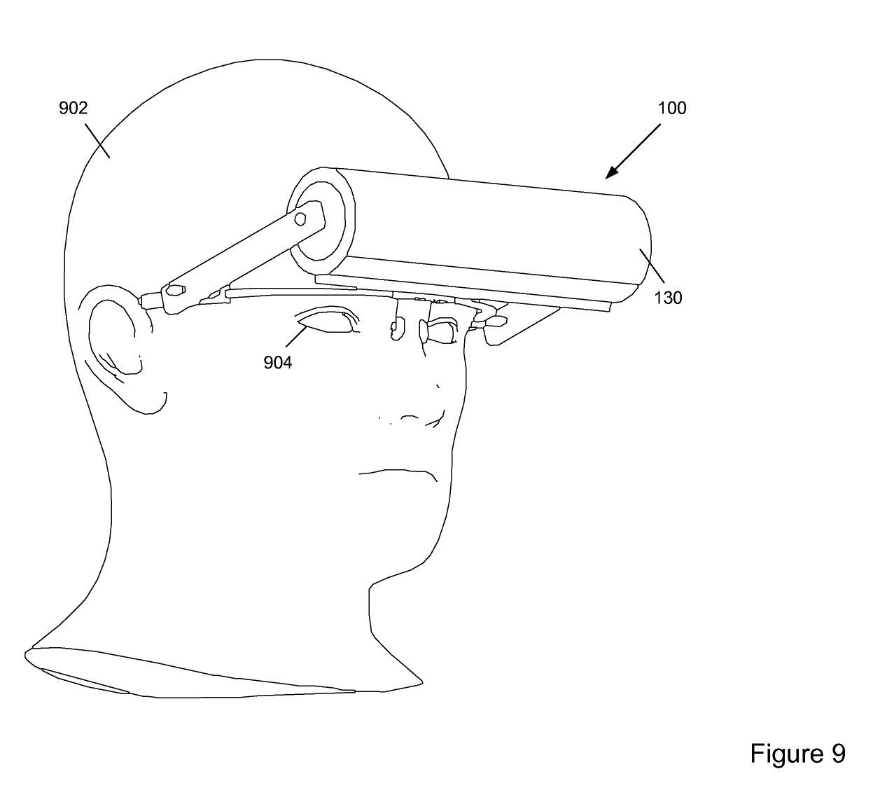

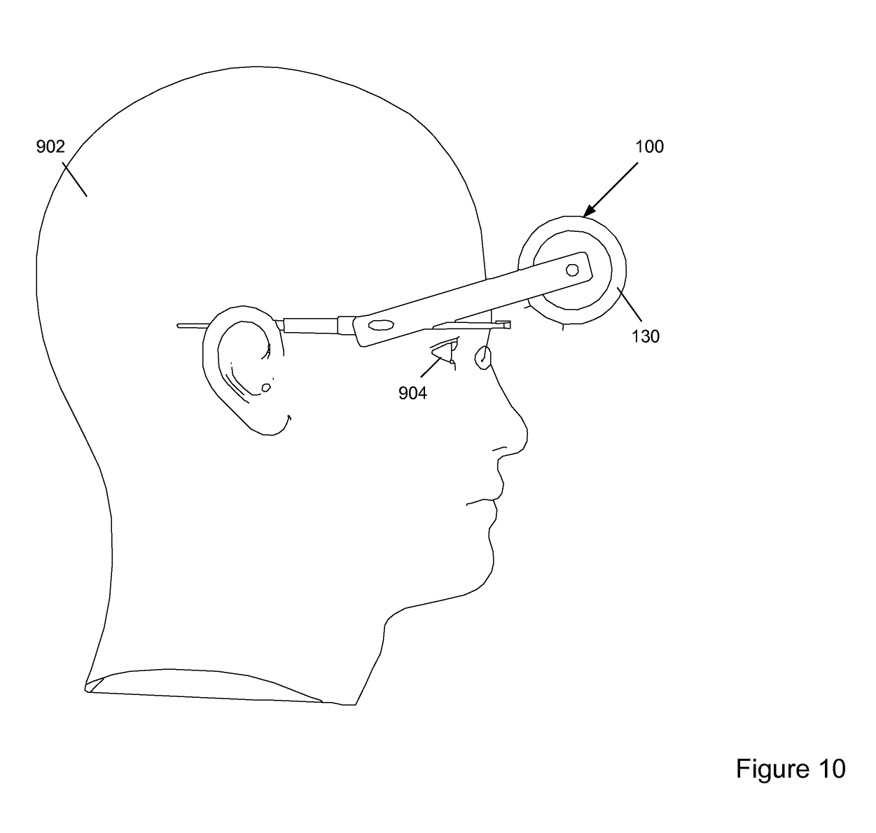

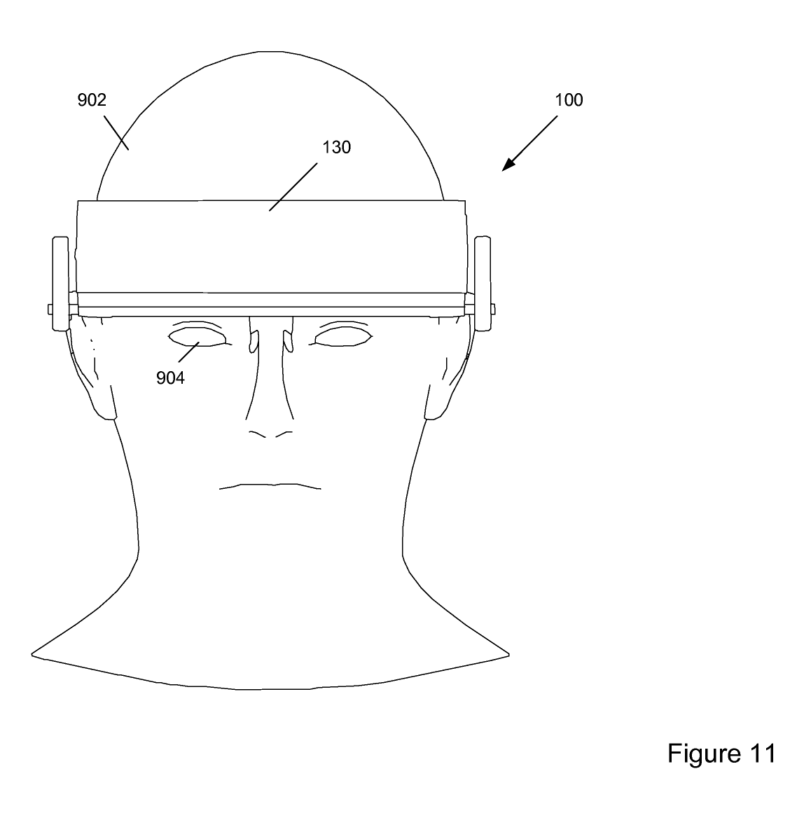

Referring now to FIGS. 9-11, the device 100 is shown as worn by an individual 902. The therapy module 130 can be positioned above a line of sight of the individual's eyes 904, so as to allow an unobstructed view as therapy is provided.

In alternative designs, modifications can be made to the light therapy device. For example, instead of using a frame, the therapy module can be mounted to the individual's head using a headband or similar structure that extends about the upper portion of the individual's head. In some examples, the headband can be made of elastic or be adjustable to assure a secure fit to the individual's head. Other configurations can be used.

In another example, a controller can be used to modify the amount of light that is emitted by the device. For example, in one embodiment, a controller can be used to modify an intensity of light provided by the LED bank. In another example including multi-colored LEDs, the controller can be used to modify which colors of LEDs lights are powered at given times. Other configurations are possible.

The various embodiments described above are provided by way of illustration only and should not be construed to limiting. Various modifications and changes that may be made to the embodiments described above without departing from the true spirit and scope of the disclosure.

What is claimed is:

1. A light therapy device, comprising:
a first module adapted to be coupled to a head of an individual; and
a second module including at least one light, the second module being coupled to the first module, and the second module powering the light;
wherein the second module is configured to be pivoted relative to the first module so that the first module is positioned relative to the individual's eyes to allow indirect light from the second module to provide indirect light therapy to the individual.

2. The light therapy device of claim 1, wherein the first module further includes a frame.

3. The light therapy device of claim 2, wherein the frame includes:
a frame front including a bridge adapted to engage a nose of the individual; and
two end pieces extending from the frame front adapted to engage the individual's ears.

4. The light therapy device of claim 3, further comprising a member positioned between the first module and the second module, wherein the member allows the second module to be pivoted relative to the first module.

5. The light therapy device of claim 1, further comprising a member positioned between the first module and the second module, wherein the member allows the second module to be pivoted relative to the first module.

6. The light therapy device of claim 1, wherein the second module further includes a cylindrical body defining an opening, and wherein the light is located within the cylindrical body.

7. The light therapy device of claim 6, wherein the light is positioned so that light emitted from the light is reflected off of the cylindrical body before exiting the opening.

8. The light therapy device of claim 7, wherein the light includes one or more light-emitting diodes.

9. The light therapy device of claim 6, wherein the cylindrical body is a general C-shape.

10. The light therapy device of claim 9, wherein the light is positioned within the cylindrical body to provide indirect light to the eyes of the individual.

11. The light therapy device of claim 1, wherein the light includes one or more light-emitting diodes.

12. A light therapy device, comprising:
a frame adapted to be coupled to a head of an individual, the frame including a frame front including a bridge adapted to engage a nose of the individual, and two end pieces extending from the frame front adapted to engage the individual's ears;
a therapy module including a cylindrical body defining an opening, and at least one light-emitting diode positioned in the cylindrical body so that light from the light-emitting diode is reflected off of the cylindrical body before exiting the opening; and
a member positioned between the frame and the therapy module, wherein the member allows the therapy module to be pivoted relative to the frame;
wherein the therapy module is positioned relative to the individual's eyes to allow light from the light-emitting diode to exit the opening in the cylindrical body to provide indirect light therapy to the individual.

13. The light therapy device of claim 12, wherein the cylindrical body is a general C-shape.

14. The light therapy device of claim 13, wherein the light includes a plurality of light-emitting diodes.

15. The light therapy device of claim 12, wherein the light includes a plurality of light-emitting diodes.

16. The light therapy device of claim 12, wherein the member includes a first member coupled to the frame that allows the therapy module to be pivoted relative to the frame.

17. The light therapy device of claim 16, wherein the member includes a second member coupled to the therapy module to allow the therapy module to rotate relative to the second member.

18. A method for providing light therapy, the method comprising:
positioning a frame on a head of an individual, the frame including a bridge adapted to engage a nose of the individual, and two end pieces extending to engage the individual's ears;
moving a therapy module that is coupled to the frame up and down relative to the frame to a desired position; and
powering at least one light-emitting diode positioned in the therapy module so that light from the light-emitting diode exits an opening formed in the therapy module to provide indirect light therapy to the individual.

19. The method of claim 18, further comprising positioning the light-emitting diode in a cylindrical member of the therapy member so that the light from the light-emitting diode is reflected off of the cylindrical member before the light exits the opening in the therapy member.

20. The method of claim 19, further comprising rotating the light therapy device to position the opening at a desired location relative to the individual's eyes.
